(19) United States
(12) Patent Application Publication
(10) Pub. No.: US 2011/0248671 A1
(43) Pub. Date: Oct. 13, 2011

(54) Power System Implantable in Eye

(75) Inventors: Cesario Dos Santos, Aliso Viejo, CA (US); Daniel Jenkins, Pomona, CA (US); Matthew Rickard, Tustin, CA (US)

(73) Assignee: ALCON RESEARCH, LTD., Forth Worth, TX (US)

(21) Appl. No.: 12/756,220

(22) Filed: Apr. 8, 2010

(51) Int. Cl. H02J 7/00; H01G 9/00; H01L 31/00; A61F 9/00; H01M 2/00; H01L 35/30

(52) U.S. Cl. 320/108; 429/163; 136/205; 136/252; 604/8; 361/434

(57) ABSTRACT

An implantable ophthalmic power system includes a power source and an enclosure. The enclosure surrounds the power source. The enclosure is configured to be implanted under the conjunctiva of the eye.

### BACKGROUND OF THE INVENTION

The present invention relates to a power system that is implantable in the eye.

Glaucoma, a group of eye diseases affecting the retina and optic nerve, is one of the leading causes of blindness worldwide. Glaucoma results when the intraocular pressure (IOP) increases to pressures above normal for prolonged periods of time. IOP can increase due to an imbalance of the production of aqueous humor and the drainage of the aqueous humor. Left untreated, an elevated IOP causes irreversible damage the optic nerve and retinal fibers resulting in a progressive, permanent loss of vision.

The eye's ciliary body epithelium constantly produces aqueous humor, the clear fluid that fills the anterior chamber of the eye (the space between the cornea and iris). The aqueous humor flows out of the anterior chamber through the uveoscleral pathways, a complex drainage system. The delicate balance between the production and drainage of aqueous humor determines the eye's IOP.

Open angle (also called chronic open angle or primary open angle) is the most common type of glaucoma. With this type, even though the anterior structures of the eye appear normal, aqueous fluid builds within the anterior chamber, causing the IOP to become elevated. Left untreated, this may result in permanent damage of the optic nerve and retina. Eye drops are generally prescribed to lower the eye pressure. In some cases, surgery is performed if the IOP cannot be adequately controlled with medical therapy.

Only about 10% of the population suffers from acute angle closure glaucoma. Acute angle closure occurs because of an abnormality of the structures in the front of the eye. In most of these cases, the space between the iris and cornea is more narrow than normal, leaving a smaller channel for the aqueous to pass through. If the flow of aqueous becomes completely blocked, the IOP rises sharply, causing a sudden angle closure attack.

Secondary glaucoma occurs as a result of another disease or problem within the eye such as: inflammation, trauma, previous surgery, diabetes, tumor, and certain medications. For this type, both the glaucoma and the underlying problem must be treated.

FIG. 1 is a diagram of the front portion of an eye that helps to explain the processes of glaucoma. In FIG. 1, representations of the lens 110, cornea 120, iris 130, ciliary bodies 140, trabecular meshwork 150, and Schlemm's canal 160 are pictured. Anatomically, the anterior chamber of the eye includes the structures that cause glaucoma. Aqueous fluid is produced by the ciliary bodies 140 that lie beneath the iris 130 and adjacent to the lens 110 in the anterior chamber. This aqueous humor washes over the lens 110 and iris 130 and flows to the drainage system located in the angle of the anterior chamber. The angle of the anterior chamber, which extends circumferentially around the eye, contains structures that allow the aqueous humor to drain. The first structure, and the one most commonly implicated in glaucoma, is the trabecular meshwork 150. The trabecular meshwork 150 extends circumferentially around the anterior chamber in the angle. The trabecular meshwork 150 seems to act as a filter, limiting the outflow of aqueous humor and providing a back pressure producing the IOP. Schlemm's canal 160 is located beyond the trabecular meshwork 150. Schlemm's canal 160 has collector channels that allow aqueous humor to flow out of the anterior chamber. The two arrows in the anterior chamber of FIG. 1 show the flow of aqueous humor from the ciliary bodies 140, over the lens 110, over the iris 130, through the trabecular meshwork 150, and into Schlemm's canal 160 and its collector channels.

A number of different implantable drainage devices (e.g. Ahmed valve, Baerveldt implant) have been developed to treat late stage glaucoma. These implants are quite large—about 12 mm by 12 mm by 1.5 mm—and are implanted under the conjunctiva of the human eye. As such, the eye can tolerate these large implants. As technology is advancing, newer glaucoma implants are being developed. It would be desirable to enhance the functionality of these implants by adding a power system. In order to power such a device, it would desirable to have a power system that is configured for implantation into the eye.

### SUMMARY OF THE INVENTION

In one embodiment consistent with the principles of the present invention, the present invention is an implantable ophthalmic power system. The power system has a power source and an enclosure. The enclosure surrounds the power source. The enclosure is configured to be implanted under the conjunctiva of the eye.

It is to be understood that both the foregoing general description and the following detailed description are exemplary and explanatory only and are intended to provide further explanation of the invention as claimed The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the front portion of an eye.

FIG. 2 is a top view of an implantable power system according to the principles of the present invention.

FIG. 3 is a top view of an implantable power system according to the principles of the present invention.

FIGS. 4A and 4B are perspective views of an implantable power system according to the principles of the present invention.

FIGS. 5A and 5B are perspective views of an implantable power system according to the principles of the present invention.

FIGS. 6A and 6B are block diagrams of an implantable capacitor array according to the principles of the present invention.

FIG. 7 is a diagram of implantable rechargeable power system with a loop antenna according to the principles of the present invention.

FIGS. 8 and 9 are diagrams of implantable rechargeable power systems that are encapsulated by a single layer according to the principles of the present invention.

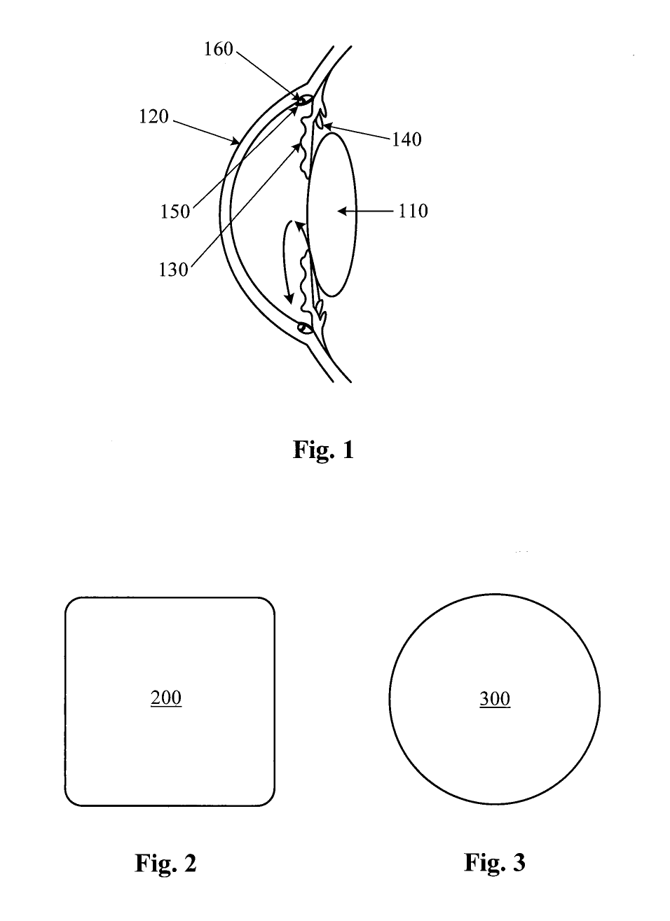

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

FIGS. 2 and 3 are top views of two exemplary implantable power systems according to the principles of the present invention. In FIG. 2, the implantable power system 200 has a generally square or rectangular shape with rounded corners. In FIG. 3, the implantable power system 300 has a generally circular or disc shape.

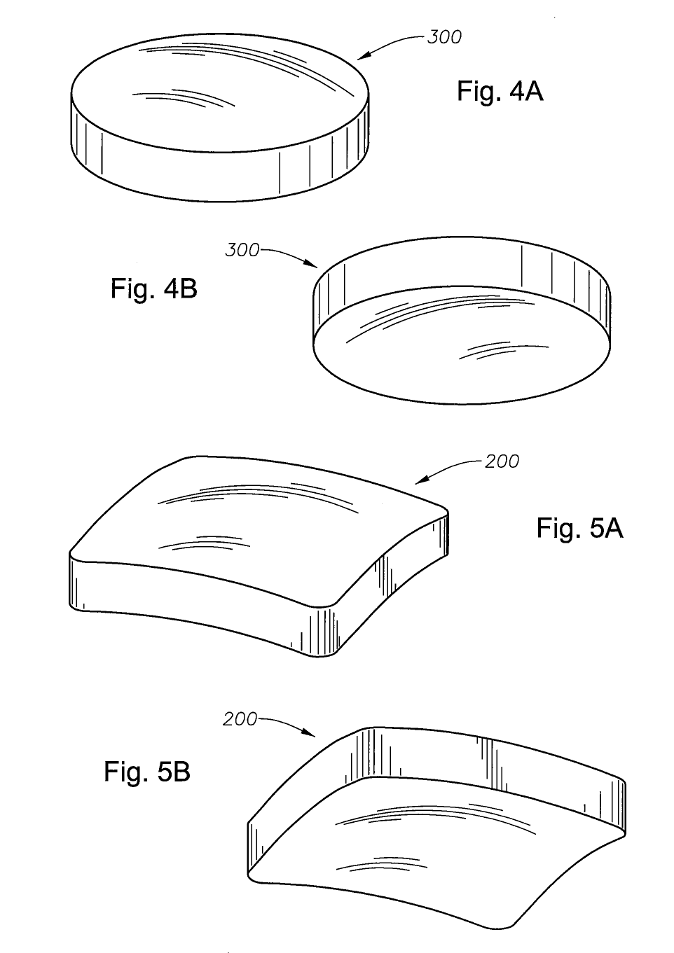

FIGS. 4A, 4B, 5A and 5B are perspective views of the implantable power supplies (200 and 300) of FIGS. 2 and 3. In FIGS. 4 and 5, the implantable power supplies 200 and 300 are curved so as to fit the curvature of the human eye.

Implantable power system 200 has dimensions of about 12 millimeters by 12 millimeters wide by 1.5 millimeters thick. In other embodiments of the present invention, the dimensions of implantable power system 200 are less than 12 millimeters by 12 millimeters wide. The thickness of implantable power system 200 is typically between one and two millimeters, although thicknesses of less than one millimeter may be achieved.

Implantable power system 300 has a diameter of about 12 millimeters and is about 1.5 millimeters thick. In other embodiments of the present invention, implantable power system 300 is less than 12 millimeters in diameter. The thickness of implantable power system 300 is typically between one and two millimeters, although thicknesses of less than one millimeter may be achieved.

Implantable power systems 200 and 300 have a curved profile that fits the curvature of the human eye. In other words, the bottom surface of implantable power system 200 or 300 rests on the surface of the sclera (when implanted under the conjunctiva). The radius of curvature is approximately 8 to 16 millimeters. In one embodiment of the present invention, the implantable power system may be made using a cross pattee configuration. A cross pattee configuration allows for the radius of curvature to be more easily implemented. In a cross pattee configuration, wedges of material are removed from a sheet of material so that when the edges of the wedges are placed adjacent to each other, a radius of curvature is approximated.

Implantable power systems 200 and 300 may be rigid or flexible. When rigid, implantable power systems 200 and 300 may be made of a biocompatible material such as stainless steel. In this manner, a stainless steel case with the above dimensions contains the components of the power system. In other embodiments of the present invention, the case may be made with any rigid material and then coated with a biocompatible material such as polypropylene or silicone. In yet other embodiments of the present invention, the case may be made directly made from a biocompatible polymeric material such as polypropylene or silicone. Since the final form factor can be very similar to existing implantable tube-to-plate drainage devices (e.g. Ahmed valve, Baerveldt implant), the packaged power system can also serve as the plate portion of such a device.

When flexible, implantable power systems 200 and 300 may be made of a biocompatible material that can be shaped to conform to the curvature of the human eye. In this case, the components inside the power systems 200 and 300 are also flexible—such as a capacitor array on a flexible substrate or a flexible thin film battery.

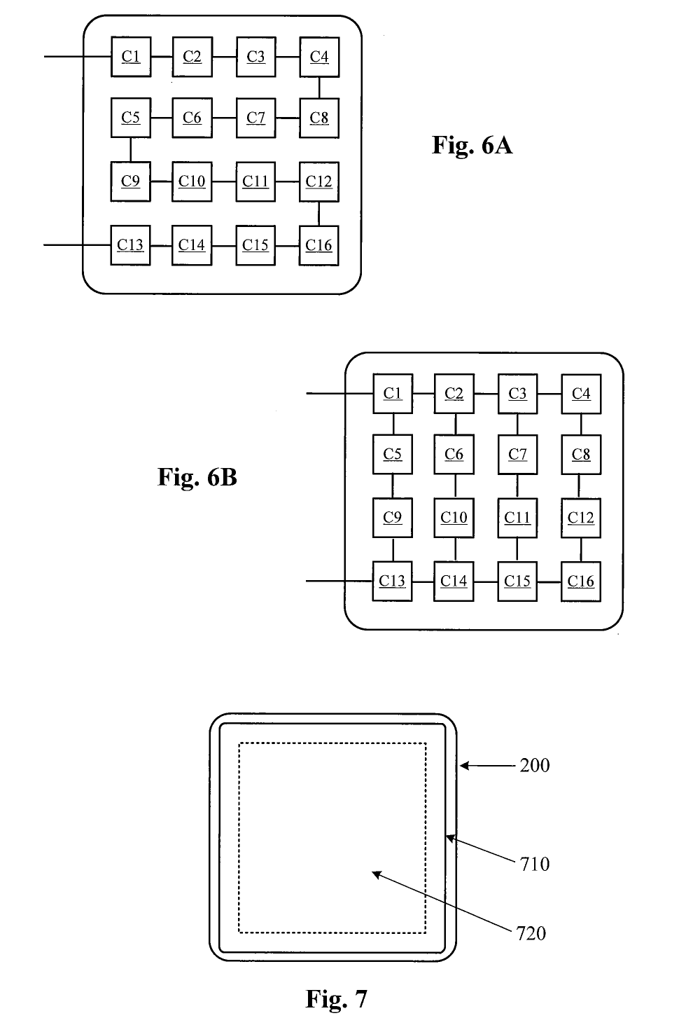

FIG. 6A is an implantable capacitor array according to the principles of the present invention. In the example of FIG. 6A, implantable power system 200 has 16 capacitors (C1-C16) connected in series. In other examples, any number of capacitors can be used, and the capacitors can be connected in series, parallel, or a hybrid of series and parallel such as that shown in FIG. 6B. The capacitor array can be planar or vertical (in which case capacitors can be stacked). For example, a four by six array of 10 microfarad capacitors (that each measure 2×1.25×1.25 mm) at four volts can store 30 mJ of energy. The size of this capacitor array is approximately 11×12×1.5 mm.

FIG. 7 is an implantable rechargeable power system with a loop antenna 710 according to the principles of the present invention. In FIG. 7, a battery 720 occupies most of the area of implantable power system 200. A loop antenna 710 is located around the periphery of the battery. The loop antenna 710 and any associated charging circuitry (not shown) function to charge battery 720. In this manner, an RF link can be used to charge battery 720. The capacitor array of FIG. 6 may also be charged in this manner as well.

In one embodiment of FIG. 7, the implantable power system 200 includes a rechargeable battery, such as a lithium ion or lithium polymer battery, although other types of batteries may be employed. In other embodiments, thin film battery technology or other type of power cell is appropriate for power system 200.

In another embodiment of the present invention a thermoelectric module can be used instead of battery 720. A thermoelectric module converts heat conducting out of the body into electrical current using the thermoelectric effect or Peltier effect. Under normal conditions, heat conducts out of the eye through the eyelid and into the air. As such, the globe of the eye is at a higher temperature that the surface of the eye that contacts the outside environment. A thermoelectric module can harness this temperature difference to create electrical current. When implanted under the conjunctive, the hot side of the thermoelectric module can be placed on the surface of the sclera, and the cold side of the module can be placed in contact with the conjunctiva. The thermoelectric module converts the temperature difference into electrical current.

In yet another embodiment of the present invention, a solar cell module can be used instead of battery 720. The solar cell module converts ambient light into electrical current. Since the eye is exposed to ambient light during most of the day, this light can be harnessed by a solar cell module. In such a case, the light collecting side of the solar cell module is implanted under the conjunctiva. Since the conjunctiva is clear, light can pass through it and strike the solar cell module. The case of the implantable power system 200, 300 can be clear as well so that light is allowed to strike the solar cell module. In another embodiment of the present invention, the light collecting face of the solar cell module is integrated into the enclosure such that it collects light that travels through the conjunctiva.

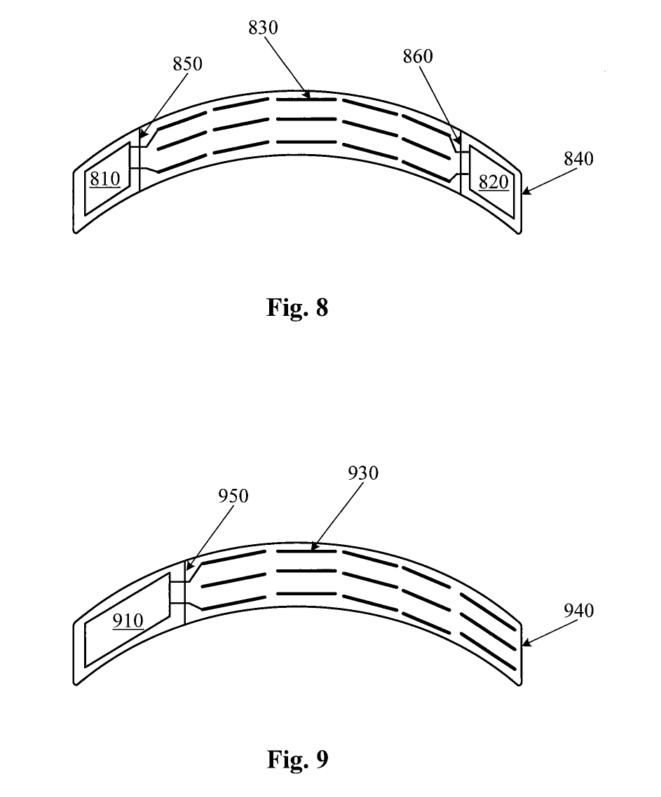

FIGS. 8 and 9 are diagrams of implantable rechargeable power systems that are encapsulated by a single layer according to the principles of the present invention. In FIG. 8, electronics modules 810 and 820 as well as capacitor array 830 are enclosed by a single layer enclosure 840. Barriers 850 and 860 separate the capacitor array 830 from the electronics modules 810 and 820. Since the capacitor array 830 contains electrolytic chemicals, it is desirable to encapsulate capacitor array 830 to protect the eye into which it is implanted and the electronics modules 810 and 820. In addition, in order to make the implant as small as possible, a single layer of material is used to encapsulate the capacitor array 830 as shown in FIG. 8. This single layer enclosure 840 is preferably made of a biocompatible material and optionally may be coated with a thin layer of silicone to ease in insertion and placement of the implantable power supply. Barriers 850 and 860 are integrated with single layer enclosure 840. Electronics modules 810 and 820 are coupled to capacitor array 830 by lead wires as shown in FIG. 8.

FIG. 9 shows an implantable power supply with a single electronics module. In FIG. 9, electronics module 910 and capacitor array 930 are enclosed by a single layer enclosure 940. Barrier 950 separates the capacitor array 930 from the electronics module 910. Since the capacitor array 930 contains electrolytic chemicals, it is desirable to encapsulate capacitor array 930 to protect the eye into which it is implanted and the electronics module 910. In addition, in order to make the implant as small as possible, a single layer of material is used to encapsulate the capacitor array 930 as shown in FIG. 9. This single layer enclosure 940 is preferably made of a biocompatible material and optionally may be coated with a thin layer of silicone to ease in insertion and placement of the implantable power supply. Barrier 950 is integrated with single layer enclosure 940. Electronics module 910 is coupled to capacitor array 930 by lead wires as shown in FIG. 9.

Electronics modules, 810, 820, and 910 function to operate the power source, in this case, capacitor arrays 830 and 930, respectively. In one example, electronics modules perform charging and discharging functions, power source maintenance functions, and the like.

The implantable power system 200, 300 is implanted into the human eye under the conjunctiva and on top of the sclera. A surgeon makes an incision in the conjunctiva near the limbus. A pocket is created by separating the conjunctiva from the sclera. The implantable power system is placed in this pocket, and the conjunctiva is sutured. In an alternate procedure, the surgeon implants the implantable power system in a pocket made in the sclera. In this case, the surgeon makes an incision in the conjunctiva and a partial incision in the sclera near the limbus. A pocket is formed in the sclera by separating layers of scleral tissue. The implantable power system is placed in the pocket, and the incisions are closed.

From the above, it may be appreciated that the present invention provides a power system that can be implanted in the eye. The present invention provides a power system that has a form factor suitable for implantation in the subconjunctival space. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An implantable ophthalmic power system comprising:
a power source; and
an enclosure surrounding the power source, the enclosure configured to be implanted under a conjunctiva of an eye.

2. The power system of claim 1 wherein the power source comprises a rechargeable battery.

3. The power system of claim 2 wherein the rechargeable battery is selected from the group consisting of: a thin film battery and a lithium polymer battery

4. The power system of claim 1 wherein the power source comprises a capacitor array.

5. The power system of claim 4 wherein the capacitor array comprises a plurality of capacitors connected in series and/or in parallel

6. The power system of claim 4 wherein the capacitor array comprises a plurality of capacitors arranged in a planar configuration.

7. The power system of claim 4 wherein the capacitor array comprises a plurality of capacitors arranged in a stacked configuration.

8. The power system of claim 1 wherein the power source comprises a thermoelectric module.

9. The power system of claim 1 wherein the power source comprises a solar cell module.

10. The power system of claim 9 wherein a light collecting face of the solar cell module is integrated into the enclosure.

11. The power system of claim 1 further comprising:
a loop antenna located around the periphery of the power source, the loop antenna coupled to the power source.

12. The power system of claim 11 wherein the loop antenna is configured to recharge the power source.

13. The power system of claim 1 wherein the enclosure has a top surface and a bottom surface, wherein the bottom surface is less than about 12 millimeters wide and less than about 12 millimeters long, wherein the distance between the top surface and the bottom surface is less than about 2 millimeters, and wherein the bottom surface has a radius of curvature of about 8 to 16 millimeters.

14. The power system of claim 1 wherein the enclosure has a top surface and a bottom surface, wherein the bottom surface has a diameter of less than about 12 millimeters, wherein the distance between the top surface and the bottom surface is less than about 2 millimeters, and wherein the bottom surface has a radius of curvature of about 8 to 16 millimeters.

15. The power system of claim 1 wherein the enclosure is made of a biocompatible material.

16. The power system of claim 1 wherein the enclosure serves as a plate portion in a glaucoma drainage device.

17. The power system of claim 1 wherein the enclosure is made of a single layer of biocompatible material.

18. The power system of claim 17 wherein a barrier separates the power source from an electronics module, and wherein the barrier is integral with the enclosure.
